(19) United States
(12) Patent Application Publication
(10) Pub. No.: US 2007/0088389 A1
(43) Pub. Date: Apr. 19, 2007

(54) Kit and method for EEA anvil placement in bariatric surgery

(75) Inventors: Brian J. Dunkin, Pinecrest, FL (US); Jose M. Martinez, Miami, FL (US)

Correspondence Address: NIXON & VANDERHYE, PC, 901 NORTH GLEBE ROAD, 11TH FLOOR, ARLINGTON, VA, 22203, US

(73) Assignee: University of Miami, Miami, FL (US)

(21) Appl. No.: 11/398,768

(22) Filed: Apr. 6, 2006

Related U.S. Application Data
(60) Provisional application No. 60/669,900, filed on Apr. 11, 2005.

(51) Int. Cl. A61B 17/04

(52) U.S. Cl. 606/219; 227/179.1

(57) ABSTRACT

A system of catheters, guidewires filaments, loops and laparoscopic instruments to simplify the placement of the anastomosis device to connect a stomach pouch to the small intestine in bariatric surgery, the surgical reduction of stomach size to treat morbid obesity.

This application claims the benefit of Provisional application No. 60/669,900, filed Apr. 11, 2005, the entire content of which is hereby incorporated by reference in this application.

### BACKGROUND OF THE INVENTION

The present invention provides a kit and method for the transoral passage and transgastric positioning of the anvil for the End-to-End Anastomotic (EEA) device in bariatric surgery.

Biatric surgery is the surgical reduction of stomach size to treat morbid obesity. Referred to as the Roux-en-Y procedure, this gastric bypass procedure promotes early satiety and malabsorption of food that is ingested. A small pouch is created from the stomach, about the size of an egg. The small intestine is divided and reconnected into a Y consisting of two limbs and a common channel to bypass the majority of the stomach as well as a portion of the small intestine. The pancreo-biliary limb is the proximal small bowel, which is maintained attached to the stomach and the duodenum and serves to drain bile, digestive enzymes, and gastric secretions. The food limb or roux limb is attached to and drains the small gastric pouch through a gastroenteral anastomosis. At the junction of these two limbs, the food mixes with the digestive juices and then proceeds along the common channel towards the colon.

Until recently, gastric by-pass has been performed as an open, single incision surgical procedure. However, laparoscopic gastric bypass techniques have been developed. The laparoscopically performed procedure is performed through a series of smaller incisions in the abdominal area. The result is decreased pain, faster recovery and more rapid return to normal activity and work.

Many surgeons create the anastomosis between the gastric pouch and the jejunum, the gastroenteral anastomosis by using the circular EEA stapler. One method of creating this anastomosis is to pass the anvil of the EEA transorally, down the esophagus and across the gastric pouch so that the tip of the anvil protrudes from the gastric wall. The anvil tip is then attached to a post emanating from the EEA hand piece, which has been introduced into the jejunum. After the anvil and hand piece are attached and brought into proximity to each other, the device is fired creating a circular stapled anastomosis. The most common method of passing the anvil involves attaching it to a nasogastric tube (NGT), which is then passed transorally down the esophagus and into the gastric pouch. A small opening is made surgically in the gastric pouch and the NGT is passed through this opening and then used to pull the anvil through the mouth, down the esophagus, and into position across the gastric pouch wall. Positioning the EEA anvil in this manner can be tedious and time-consuming causing many surgeons to abandon this approach.

### BRIEF DESCRIPTION OF THE INVENTION

The invention provides a kit and method for facilitating and simplifying anvil passage and placement. In an embodiment of the invention, a large caliber introducer is passed transorally into the gastric pouch. The introducer has a central lumen large enough to accommodate, e.g., a catheter knife assembly which may then in turn be used to form a small hole through an appropriate area of the gastric pouch wall. The catheter of the catheter knife assembly is adapted to be positioned across the gastric wall after the incision is made. Next, a filament is introduced through the catheter and into the peritoneal space where it is grasped by the surgeon. The introducer and catheter knife assembly may then be removed and the anvil secured to the filament for being fed transorally to the incision/perforation in the gastric pouch, through which it may extend for engagement with the EEA handpiece.

Thus, the invention may be embodied in a method for the passage and positioning of an anvil component of an end-to-end anastomotic device comprising: providing an introducer comprising a tube having distal and proximal ends; disposing said introducer to pass through a natural orifice to a first target wall segment of a first biological dust or organ in which the introducer is disposed; viewing an outer surface of the target wall segment and visually determining a location of said introducer with said first biological duct or organ; advancing a catheter knife assembly through said introducer to said first target wall segment; using said catheter knife assembly to form an opening through said first target wall segment; advancing the catheter knife assembly through said first target wall segment; threading a filament through the catheter of said catheter knife assembly; removing said catheter and said introducer while retaining said filament extending through said first target wall segment; securing said anvil component to a proximal end of said filament; and pulling a distal end of said filament so as to guide said anvil through said natural orifice and to said opening to align said anvil in surrounding relation to said opening.

In an example embodiment, the method may further comprise inserting a second, handheld component of said anastomotic device to extend through an opening in a second target wall segment in a second biological duct or organ to be attached to said first target wall segment; aligning and attaching said first and second components and actuating said anastomotic device to form an anastomosis between said first and second target wall segments and withdrawing said anastomotic device.

The invention may also be embodied in a kit for placement of one part of a two part anastomotic device comprising: an introducer; a catheter knife assembly for placement through said introducer; and a filament for threading through said catheter of said catheter knife assembly.

Modifications may be made to the components to further simplify or facilitate the procedure. For example, a fiber optic light may be added to the introducer for transilluminating the gastric pouch to guide and determine placement of its tip for passage of the catheter knife assembly. A second alternative is to make the catheter of large enough diameter to accommodate the filament without removal of the knife itself. This would save a step in the procedure. Finally, the inner catheter could be used in the absence of an introducer tube by introducing it through an introducer in the form of a gastroscope, so that the procedure could be performed under direct visualization.

### BRIEF DESCRIPTION OF THE DRAWINGS

FIG. 1 is a schematic view of a proximally transected stomach with small intestine severed in accordance with a gastric bypass procedure;

FIG. 2 is a view similar to FIG. 1 illustrating the reconnection of a proximal limb of the small intestine downstream;

FIG. 3 is a schematic illustration of an introducer disposed in a gastric pouch as a first step in a method embodying the invention;

FIG. 4 illustrates a needle knife forming an opening in the gastric pouch in an embodiment of the invention;

FIG. 5 depicts the advancement of the needle knife catheter through the formed opening;

FIG. 6 illustrates the disposition of a filament through the catheter, after removal of the needle knife from the catheter in this example embodiment;

FIG. 7 is a view of the gastric pouch from another angle, illustrating the threaded filament with introducer and catheter knife assembly removed;

FIG. 8 is a schematic illustration showing an anvil attached to the proximal end of the threaded filament;

FIG. 9 is a view similar to FIG. 7 illustrating the threading of the anvil to the target wall segment;

FIG. 10 illustrates the second component of the anastomotic device positioned for attachment to the first component or anvil;

FIG. 11 is a view similar to FIG. 10 illustrating the components attached and ready for EEA device actuation;

FIG. 12 is an illustration of the formed anastomosis;

FIG. 13 is a view of the gastrointestinal configuration at the conclusion of the procedure.

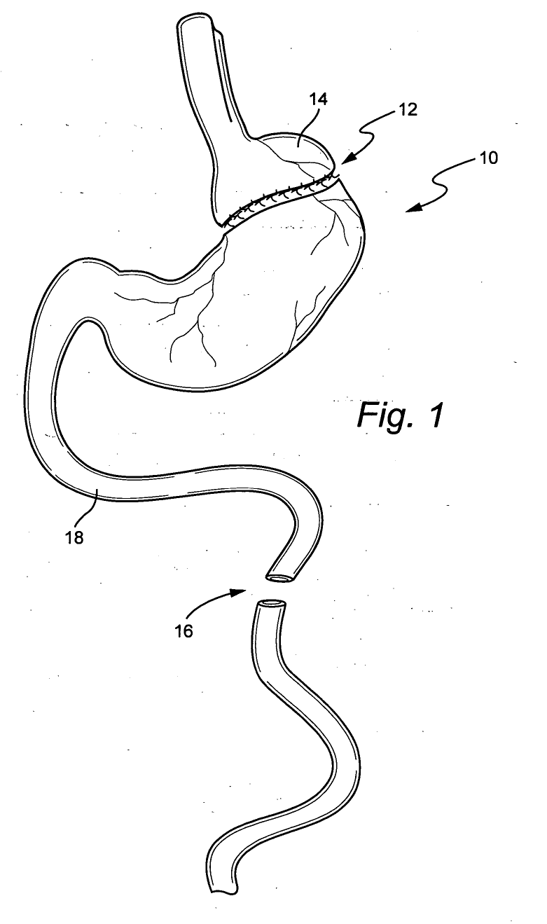

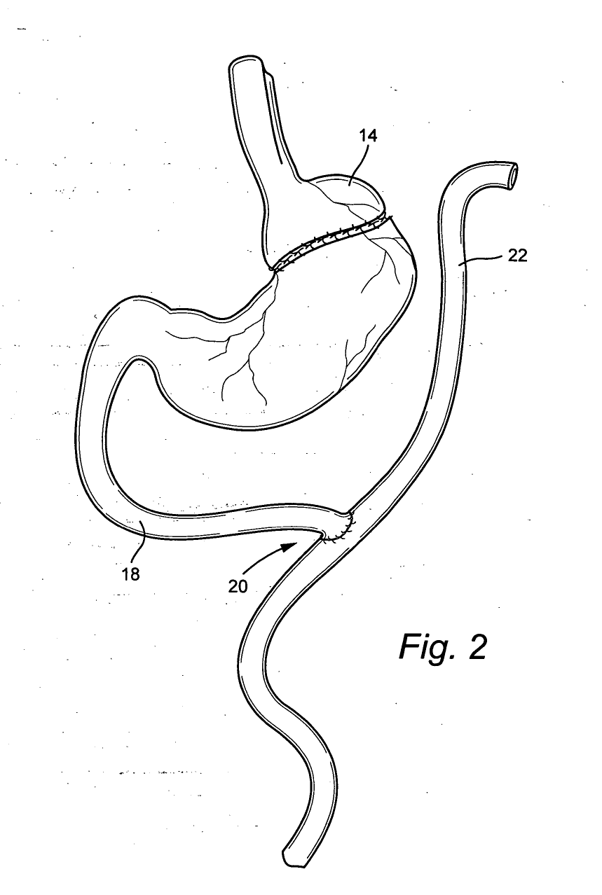

### DETAILED DESCRIPTION OF THE INVENTION

The kit and method of the invention will be described hereinbelow in connection with a gastric bypass, or Roux-en-Y procedure, it being understood that gastric bypass is just one procedure in which the kit of the invention may be used to advantage.

As mentioned above, laparoscopic gastric bypass is now a well-established procedure for the surgical management of morbid obesity. As a first step in the bypass procedure, the stomach 10 is proximally transected as at 12 with a linear stapler to create a small gastric pouch 14 having a volume of about 15 to 30 ml. The small intestine is then reconfigured into a Y.

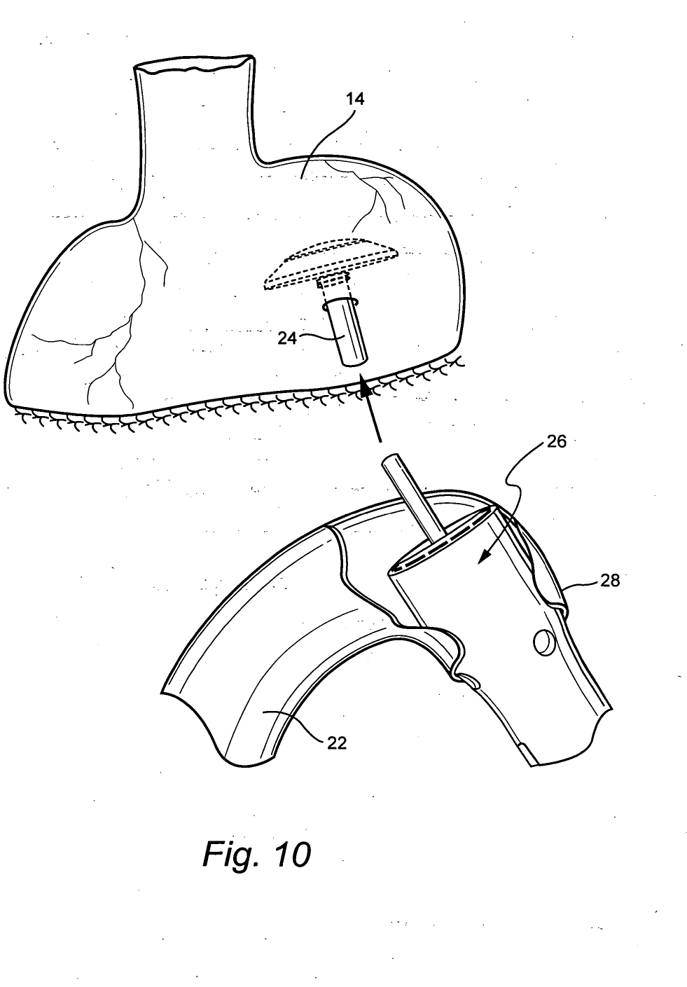

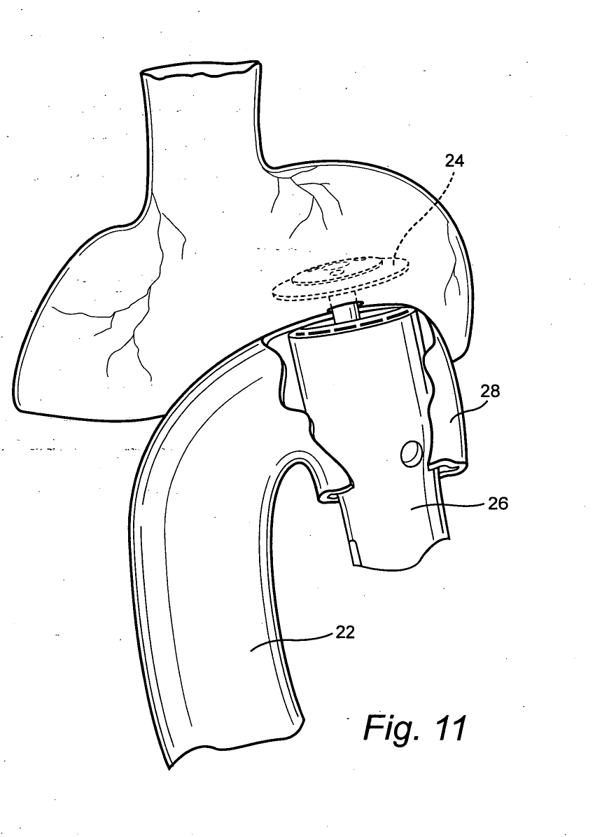

As illustrated, the small intestine is severed as at 16 to create the first limb 18 of the reconfigured small intestine. This proximal portion 18 of the small intestine remains attached to the stomach and duodenum. This limb is sometimes called the pancreo-biliary conduit or limb 18. It serves to drain bile, digestive enzymes and gastric secretions to facilitate digestion downstream. The distal end of the first limb is sutured to the downstream segment of small intestine as at 20 to join the internal passages of the respective limbs. The food limb, sometimes referred to as the Roux limb 22 is to be attached to drain the proximal gastric pouch 14. It is the attachment of the Roux limb 22 to the gastric pouch 14 which is the primary subject of this invention. Many surgeons create the gastroenteral anastomosis, the junction between the gastric pouch and the Roux limb, using a circular EEA stapler. The EEA stapler is comprised of an anvil segment 24 and a handheld stapler 26. The anvil segment 24 must be disposed to extend through the gastric pouch wall and the hand held segment is disposed through the free end 28 of the Roux limb 22 as illustrated in FIGS. 10-11. The present invention thus relates in particular to a kit for more easily placing the anvil 24 to extend through the gastric pouch 14 at a determined location for ultimate attachment to the handheld EEA device 26.

The anvil must be placed in the gastric pouch transorally. Meanwhile the gastric pouch is very small and the anvil must be properly disposed with respect to the pouch wall to provide the desired connection to the Roux limb. To facilitate anvil placement, the invention provides a kit including an introducer 30, a catheter knife assembly 40 and a filament 50 such as a looped wire for threading the anvil 24 to a target portion 32 of the gastric pouch wall 34.

The introducer 30 is a small gauge catheter that is introduced through the esophagus 36. In an exemplary embodiment, the introducer catheter has an outside diameter of about 12 mm (36 French) and ends in a tapered, narrowed tip 38. In accordance with an exemplary method embodying the invention, the introducer is disposed through the esophagus, inserted into the gastric pouch 14 and urged against the wall 34 of the gastric pouch so that the surgeon can endoscopically visualize and determine a location of the introducer tip 38 from within the peritoneal cavity, outside the gastric pouch. As an alternative, a fiber optic element(s) can be disposed to illuminate the tip of the introducer so that the illuminated tip can be identified from the outside of the gastric pouch. As an alternative to providing a catheter introducer, an endoscope having a passage for receiving a catheter knife assembly, as described hereinbelow, may be adopted. However, an introducer catheter having a relatively pointed tip is preferred to localize the target site for the anvil and to reduce the cost associated with the procedure.

Once the introducer has been disposed to identify and align with a target portion 32 of the gastric pouch, a catheter knife assembly 40, such as a needle tipped catheter, is inserted or threaded through the introducer 30. The needle tipped catheter is used to form a small opening 42 in the wall 34 of the gastric pouch. In an exemplary embodiment a cautery needle tipped catheter is used but the cautery function may be considered optional and, therefore, may be omitted without departing from the invention. The tip of the catheter body 44 is advanced through the opening 42 in the gastric wall into the peritoneal space. The needle tip 46 may thereafter be removed from the catheter body 44 and a filament 50 for threading the anvil 24 disposed through the catheter body 44 in its stead. In an example embodiment a double looped wire is provided as the filament 50.

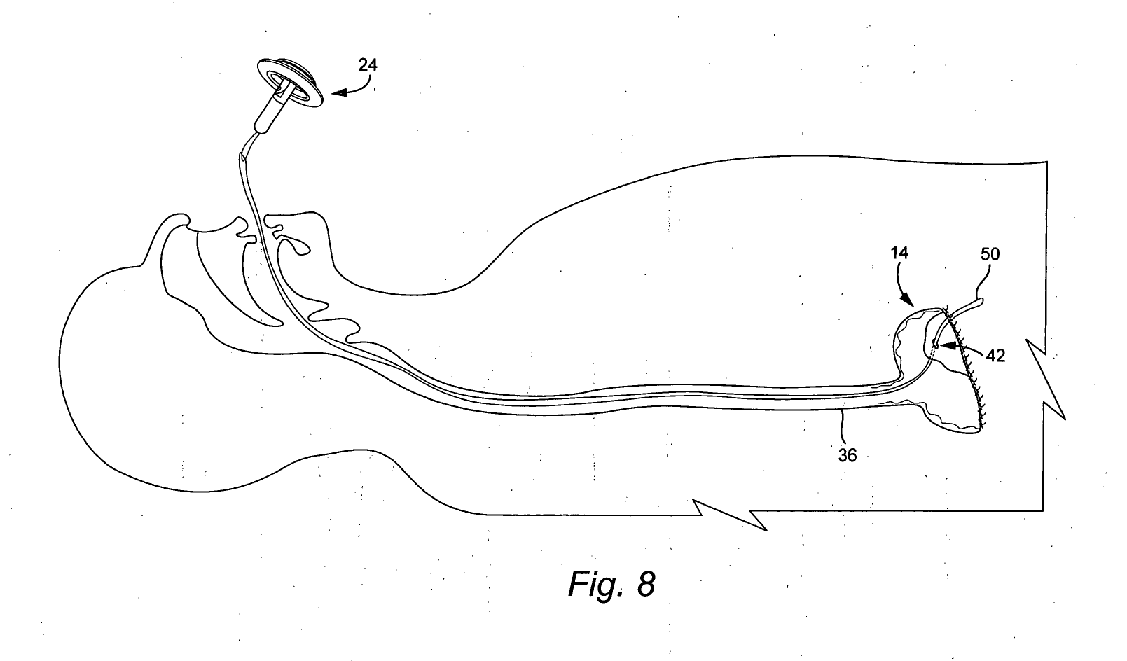

The introducer 30 and catheter body 44 may then be removed and the proximal end 52 of the filament 50, which is disposed outside the patient's mouth, may be secured to the tip 54 of the anvil 24. By pulling the filament 50 from the peritoneal cavity, with suitable endoscopic instruments or manually, the anvil 24 is pulled into position (FIGS. 8 and 9) and the feed filament 50 can be removed. The anvil will then be disposed through the opening 42 in the gastric pouch wall and disposed for attachment to the EEA hand piece 26. The EEA may then be closed and fired to create the circular anastomosis 54 and the EEA is removed. The result is a reconfigured small intestine and minuscule gastric pouch to complete the gastric bypass procedure.

As will be appreciated, the kit provided in accordance with the invention facilitates directing the anvil to a target portion of the gastric pouch wall and thereafter enables rapid feed and alignment of the anvil to facilitate the rapid formation of the anastomosis to the Roux limb.

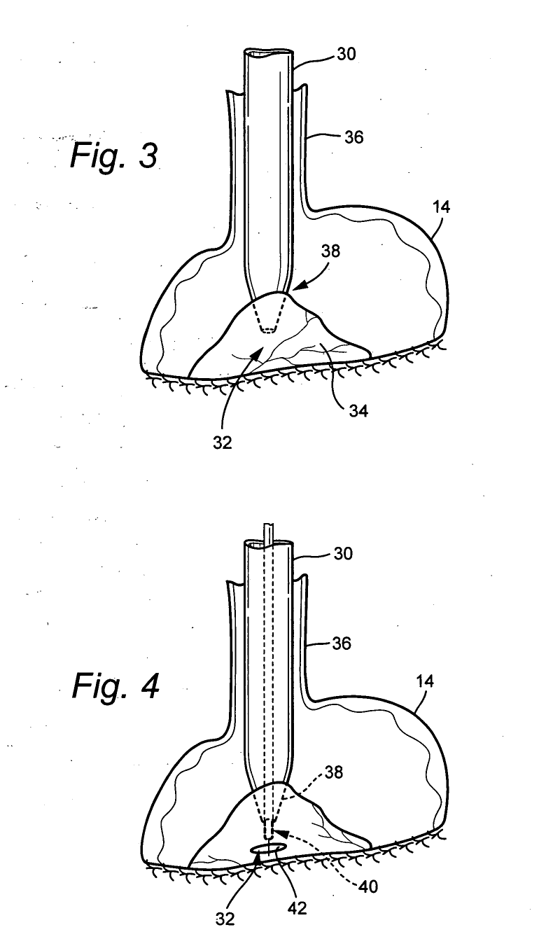

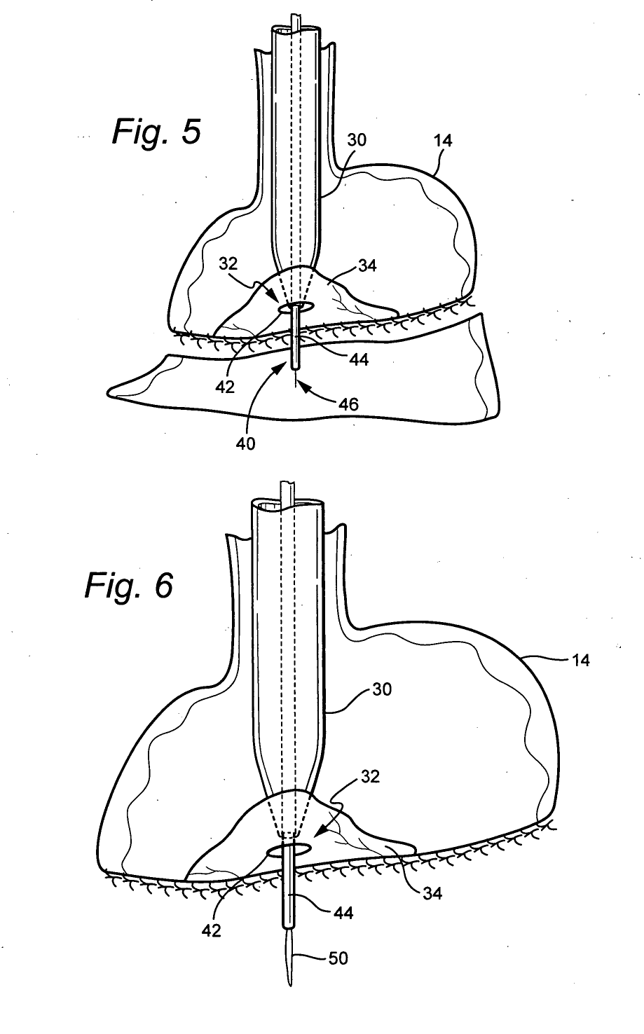

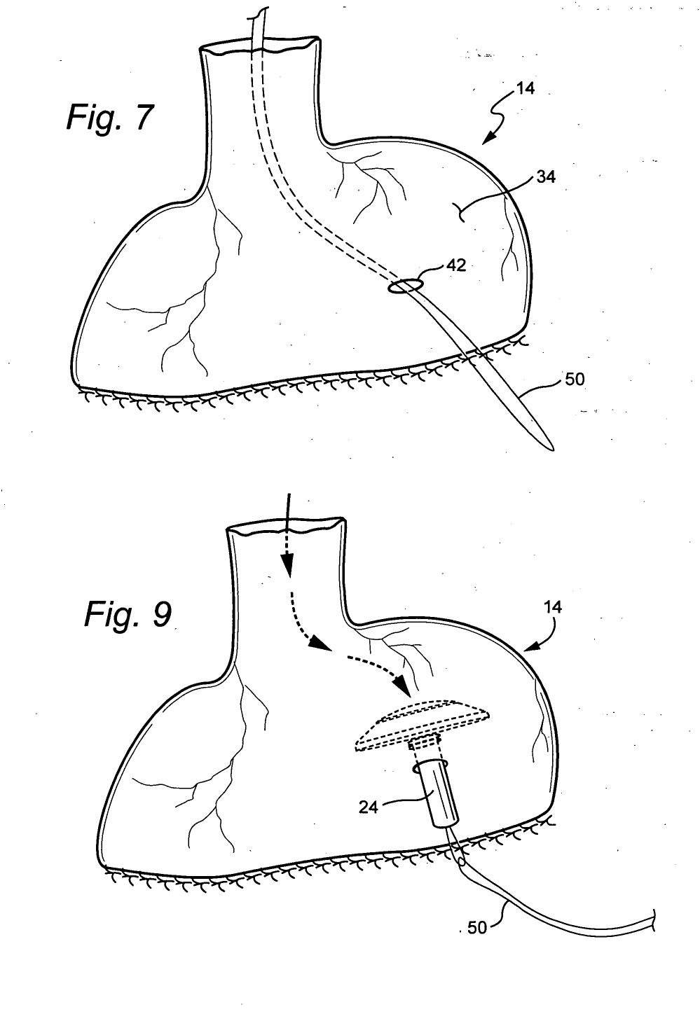

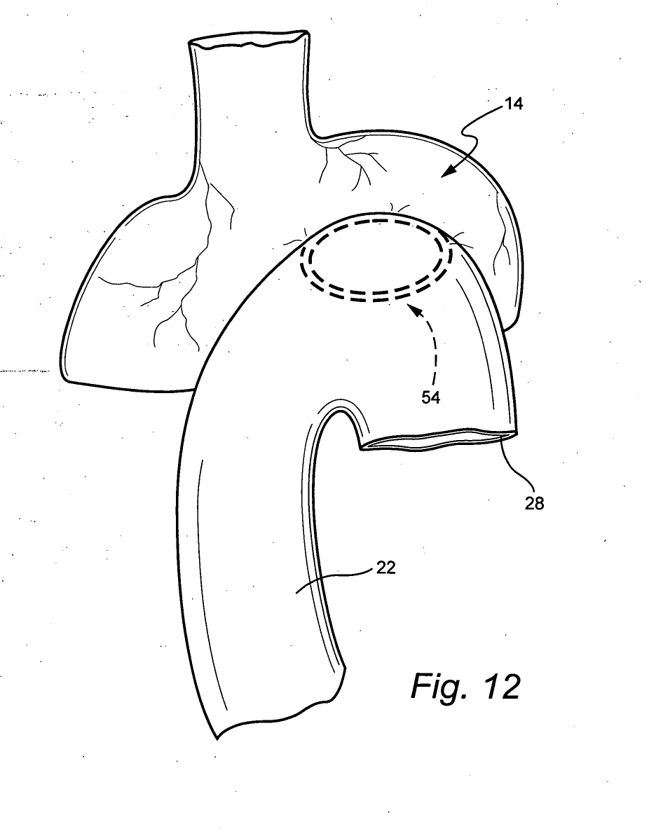

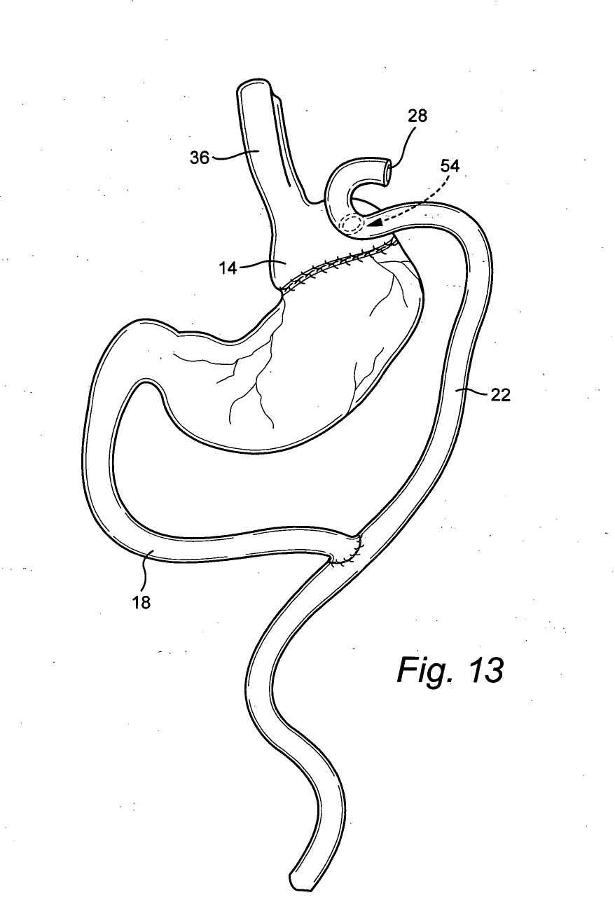

In FIGS. 3-9 and 10-12, the portion of the stomach below the staple line has been omitted. It is to be understood that the gastric pouch may be severed and separated from the remainder of the stomach adjacent the staple line or the gastric pouch may remain attached to the remainder of the stomach without modifying the procedure for anvil placement described herein.

Although in the illustrated embodiment, the needle 46 of the needle tipped catheter 40 is removed in advance of feeding the looped wire filament 50, it is to be understood that as an alternative, a needle tipped catheter may be provided that is large enough to accommodate the looped wire through a conduit thereof, so that the removal of the needle tip 46 may not be necessary to accomplish the method of the invention.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method for the passage and positioning of an anvil component of an end-to-end anastomotic device comprising:
providing an introducer comprising a tube having distal and proximal ends;
disposing said introducer to pass through a natural orifice to a first target wall segment of a first biological dust or organ in which the introducer is disposed;
viewing an outer surface of the target wall segment and visually determining a location of said introducer with said first biological duct or organ;
advancing a catheter knife assembly through said introducer to said first target wall segment;
using said catheter knife assembly to form an opening through said first target wall segment;
advancing the catheter knife assembly through said first target wall segment;
threading a filament through the catheter of said catheter knife assembly;
removing said catheter and said introducer while retaining said filament extending through said first target wall segment;
securing said anvil component to a proximal end of said filament; and
pulling a distal end of said filament so as to guide said anvil through said natural orifice and to said opening to align said anvil in surrounding relation to said opening.

2. A method as in claim 1, further comprising inserting a second, handheld component of said anastomotic device to extend through an opening in a second target wall segment in a second biological duct or organ to be attached to said first target wall segment;
aligning and attaching said first and second components and actuating said anastomotic device to form an anastomosis between said first and second target wall segments and withdrawing said anastomotic device.

3. A method as in claim 1, further comprising removing a needle component from said catheter knife assembly before said threading step.

4. A method as in claim 1, wherein said introducer has a tapered tip.

5. A method as in claim 1, wherein said visually determining a location of said introducer comprises urging said introducer against said first target wall segment.

6. A method as in claim 1, wherein said visually determining comprises illuminating at least a tip with said introducer to visualize the introducer.

7. A method as in claim 2, wherein said first target wall segment comprises a portion of the stomach wall and said second target wall segment comprises a portion of the small intestine.

8. A kit for placement of one part of a two part anastomotic device comprising:
an introducer;
a catheter knife assembly for placement through said introducer; and
a filament for threading through said catheter of said catheter knife assembly.

9. A kit as in claim 8, wherein said introducer has a tapered distal end.

10. A kit as in claim 8, wherein said introducer comprises a catheter having an outside diameter of about 12 mm.

11. A kit as in claim 8, further including fiber optic components for illuminating a tip of said introducer.

12. A kit as in claim 8, wherein said catheter knife assembly includes a lumen for receiving said filament.

13. A kit as in claim 8, wherein said filament comprises a double looped wire.
